(19) United States
(12) Patent Application Publication
(10) Pub. No.: US 2003/0212332 A1
(43) Pub. Date: Nov. 13, 2003

(54) Disposable ultrasonic soft tissue cutting and coagulation systems

(76) Inventors: Paul Fenton, Marblehead, MA (US); Francis Harrington, Peabody, MA (US); Paul Westhaver, Newburyport, MA (US)

Correspondence Address: Mark G. Lappin, McDermott, Will & Emery, 28 State Street, Boston, MA, 02109, US

(21) Appl. No.: 10/437,102

(22) Filed: May 13, 2003

Related U.S. Application Data
(60) Provisional application No. 60/380,232, filed on May 13, 2002.

(51) Int. Cl. A61B 8/14

(52) U.S. Cl. 600/459; 601/2

(57) ABSTRACT

An ultrasonic surgical system is presented that is economical to produce and utilize, by including at least one disposable component. The ultrasonic surgical system includes an ultrasonic transducer for converting electric signals into ultrasonic vibrations. An ultrasonic transmission coupler is connected to the transducer, so as to receive the ultrasonic vibrations from the transducer. An ultrasonic vibration element is coupled to the distal end of the ultrasonic transmission coupler. At least one of the ultrasonic transducer, the ultrasonic transmission coupler, and the ultrasonic vibration element is disposable. The components of the ultrasonic surgical system are not precision-cut, and are adapted to be press-fit onto each other. The ultrasonic surgical system may be tuned to a desired resonant frequency by varying the lengths of the disposable components.

### CROSS-REFERENCE TO RELATED APPLICATIONS

The present application claims priority to provisional U.S. patent application Ser. No. 60/380,232, filed on May 13, 2002, which is assigned to the assignee of the present application and incorporated herein by reference.

### STATEMENT REGARDING FEDERALLY SPONSORED RESEARCH

Not Applicable

### REFERENCE TO MICROFICHE APPENDIX

Not Applicable

### BACKGROUND OF THE INVENTION

It has long been recognized that ultrasonic instruments are useful for the safe and effective treatment of many medical conditions. In particular, ultrasonic surgical instruments have for many years been used for soft tissue cutting and coagulation. Ultrasonic instruments are advantageous because they may be used to cut and/or coagulate organic tissue using energy in the form of mechanical vibrations transmitted to a distal vibrating member, at ultrasonic frequencies.

Typically, these ultrasonic instruments include ultrasonic transducers which convert the electric energy supplied by a generator into ultrasonic frequency vibratory energy, which can then be applied to the tissue of a patient. The transducers are typically enclosed within a handpiece or a transducer sheath. Ultrasonic surgical instruments use relatively high-power, low-frequency vibratory energy, typically at a frequency range of about 20 kHz to about 100 kHz.

An ultrasonic probe connected to the transducers typically includes an elongated ultrasonic transmission coupler, and an ultrasonic element (for example an ultrasonic surgical blade) mounted to the distal end of the coupler. The coupler may be enclosed within an elongated tubular shaft. The coupler transmits the ultrasonic vibrations, generated by the transducers, to the ultrasonic element. The ultrasonic element is thereby made to vibrate at ultrasonic frequencies. The ultrasonically vibrating element is then applied to the tissue, in order to transmit ultrasonic energy to the tissue. The ultrasonic vibrations may be transmitted to the tissue at suitable energy levels. In this way, the contacted tissue can be cut or coagulated. Ultrasonic surgical systems offer a number of advantages over conventional surgical systems, for example reduction of bleeding and trauma.

The mechanism through which the ultrasonic element and the tissue interact, i.e. the physics of ultrasonic soft tissue cutting and coagulation, is not completely understood, however various explanations have been provided by researchers over the years. These explanations include descriptions of mechanical effects and thermal effects. The mechanical viewpoint states that the vibrating tip of the ultrasonic probe generates short-range forces and pressures, which are sufficient to dislodge cells in the tissue, and break up the tissue structures. Various types of forces are postulated as contributing to the rupture of the tissue layer, for example the impact forces resulting from the direct contact of the vibrating tip with tissue, and the shear forces that are the result of the differences in force levels across tissue boundaries. Some energy may be lost due to frictional heating, and by the heating caused by the absorption of acoustic energy by tissue.

Thermal effects may include frictional heat, generated by the ultrasonically vibrating tip, in an amount sufficient to melt a portion of the contacted tissue. Alternatively, the tissue may absorb the vibratory energy, which it then converts into heat. The generated heat may be used to coagulate a blood vessel, by way of example. Other effects that have been postulated in order to explain the probe-tissue interaction include cavitational effects. The cavitation viewpoint postulates that the coupling of ultrasonic energy onto tissue results in the occurrence of cavitation in tissue, namely the formation of gas or vapor-filled cavities or bubbles within the tissue, which may oscillate and propagate. A combination of mechanical, thermal, and cavitational effects may result in the desired surgical outcomes, such as cutting and coagulation.

A number of ultrasonic soft tissue cutting and coagulating systems have been disclosed in the prior art. For example, U.S. Pat. No. 5,322,055 (the “'055 patent”), assigned on its face to Ultracision, Inc., discloses ultrasonic surgical instruments having a non-vibrating clamp for pressing tissue against an ultrasonically vibrating blade, for cutting, coagulating, and blunt-dissecting of tissue. When ultrasonically activated, the blade undergoes longitudinal mode vibrations, parallel to the blade edge. U.S. Pat. No. 6,036,667 (the “'667 patent”), assigned on its face to United States Surgical Corporation and to Misonix Incorporated, discloses an ultrasonic dissection and coagulation system. The ultrasonic system includes an ultrasonic cutting blade, and a clamp member for clamping tissue in conjunction with the blade. The blade has a cutting surface that is angled with respect to the longitudinal axis of vibration.

U.S. Pat. No. 6,056,735 (the “'735 patent”), assigned on its face to Olympus Optical Co., Ltd., relates to ultrasonic treatment systems, including endoscopic systems and aspiration systems, for treating living tissue. The '735 patent features an ultrasonic treatment system including a probe which conveys ultrasonic vibrations to a stationary distal member. The stationary distal member cooperate with a movable holding member to clamp or free tissue, when manipulated by a scissors-like manipulating means.

In prior art ultrasonic surgical systems, the constituent parts, such as the ultrasonic transducer, the transducer sheath, the ultrasound transmission coupler, and the ultrasonic surgical blade, are generally precision-cut, and therefore not disposable or replaceable. By way of example, these constituent parts may be precision-cut in order to place a vibratory node (or antinode) of the instrument at the desired or necessary location along the instrument, i.e. in order to tune the vibrations of the ultrasonic instrument at desired frequencies. Using precision-cut component parts allows desired features (for example, the desired frequencies of the ultrasonic vibrations), which are specific to the particular surgical procedure being use or the particular tissue being treated, to be incorporated into the surgical system. However, using precision-cut component parts increases the cost of manufacturing and assembling the ultrasonic surgical instruments.

There is therefore a need for low cost devices that can be used for ultrasound surgery, and that are formed of inexpensive, disposable, and replaceable component parts.

It is an object of this invention to provide an improved ultrasonic surgical system that is economical to produce and utilize, and that contains one or more components that is disposable after use.

### SUMMARY OF THE INVENTION

The present invention is directed to ultrasonic surgical systems that are inexpensive to manufacture and utilize, and include at least one disposable and replaceable component. The costs involved in manufacturing and using the ultrasonic surgical systems are lowered, by avoiding precision-cut component parts.

An ultrasonic surgical system constructed in accordance with the present invention includes an ultrasonic transducer for converting electric signals into ultrasonic vibrations, and an ultrasonic transmission coupler connected to the transducer so as to receive the ultrasonic vibrations from the transducer. The transmission coupler is preferably elongated, and is adapted to transmit the ultrasonic vibrations from a proximal end thereof to a distal end thereof. An ultrasonic vibration element is coupled to the distal end of the ultrasonic transmission coupler. The ultrasonic vibration element may be a surgical blade, for example.

The ultrasonic surgical system may include an ultrasonic transducer sheath for enclosing the ultrasonic transducer. The ultrasonic transmission coupler may also be enclosed within an elongated tubular sheath.

In the present invention, at least one of the ultrasonic transducer, the ultrasonic transmission coupler, the ultrasonic vibration element, the ultrasonic transducer sheath, and the elongated tubular sheath for enclosing the ultrasonic coupler, is disposable.

In one embodiment, the entire ultrasonic surgical system may be disposable, being formed solely from disposable constituent components.

The ultrasonic surgical system may be characterized by a resonant frequency. The disposable components may be made of constant cross-section material, and be adapted to have lengths that can be varied so that the resulting ultrasonic surgical system achieves a desired resonant frequency.

Suitable materials for a disposable ultrasonic transducer may include, but are not limited to, piezoelectric materials, piezoceramic materials, and nickel. Suitable materials for a disposable ultrasonic vibration element (for example a disposable ultrasonic surgical blade) may include, but are not limited to, plastics, ceramics, polymers, polycarbonates, metals, and plastic-metal alloys.

The ultrasonic surgical system may include a control unit for controlling the amplitude of the ultrasonic vibrations generated by the ultrasonic surgical system. The control unit may also control the frequency and/or duration of the ultrasonic vibrations. Preferably, the control unit is a hand-controlled unit, and may also be disposable.

### BRIEF DESCRIPTION OF THE DRAWINGS

The invention can be more fully understood by referring to the following detailed description taken in conjunction with the accompanying drawings, in which:

FIG. 1 illustrates an overall schematic view of an ultrasonic surgical system, constructed in accordance with one embodiment of the present invention.

FIG. 2 provides a schematic illustration of an ultrasonic surgical system whose resonant frequency is tunable by varying the length of one or more of its constituent disposable components.

FIG. 3 provides a schematic illustration of an ultrasonic surgical system having a manually controllable control unit for controlling the duration and/or frequency and/or amplitude of the ultrasonic vibrations.

### DETAILED DESCRIPTION

The present invention features ultrasonic surgical systems that include at least one disposable component part. The disposable component parts may include, but are not limited to, an ultrasonic transducer, an ultrasonic transmission coupler, an ultrasonic vibration element (for example an ultrasonic surgical blade), and an ultrasonic transducer sheath. By using disposable component parts that are replaceable after use, and that are not precision-cut, the ultrasonic surgical systems of the present invention are much more economical to produce and to utilize, as compared to prior art ultrasonic surgical systems.

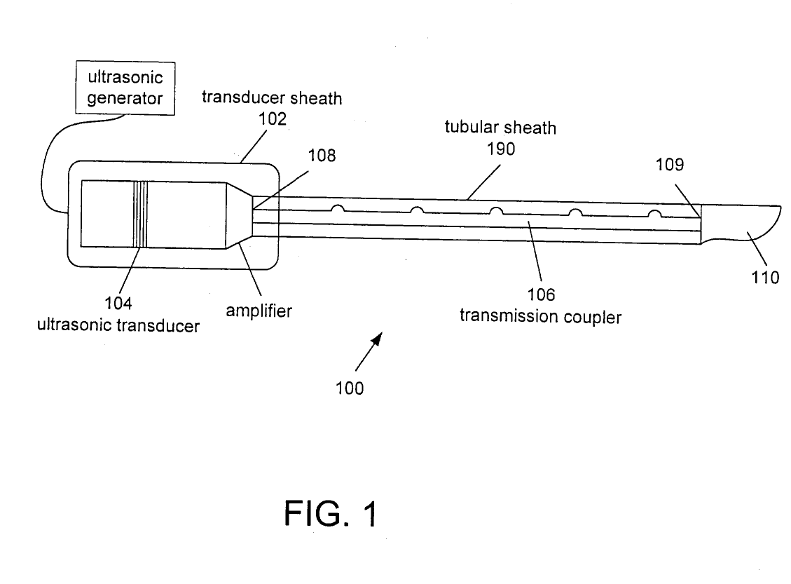

FIG. 1 illustrates an overall schematic view of an ultrasonic surgical system 100, constructed in accordance with one embodiment of the present invention. The system 100 includes an ultrasonic transducer sheath 102 that encloses one or more ultrasonic transducers 104. An ultrasonic generator is connected to the transducer sheath 102, and supplies electric energy. The transducers 104 convert the supplied electric energy into ultrasonic frequency vibratory energy. The frequency range at which the system 100 operates is typically between about 20 kHz and about 100 kHz, and the electric power supplied by the ultrasonic generator is typically between about 100 W to about 150 W, although other frequencies and power levels can be used. The ultrasonic transducers 104 may be made of piezoelectric material, or may be made of other materials, such as nickel, that are capable of converting electric energy into vibratory energy. The transducer sheath 102 may also enclose an amplifier, for example an acoustic horn, which amplifies the mechanical vibrations generated by the ultrasonic transducers 104.

An elongated ultrasonic transmission coupler 106 is connected to the transducer sheath 102. In one embodiment, the transmission coupler 106 has a proximal end 108 and a distal end 109, and is connected to the transducer sheath 102 at the proximal end. The ultrasonic transmission coupler 106 transmits the ultrasonic vibratory energy, received from the transducers 104, from its proximal 108 end to its distal end 109. In one embodiment, a tubular sheath 190 may enclose the transmission coupler 106.

In the illustrated embodiment, an ultrasonic vibration element 110 is connected to the distal end 109 of the elongated transmission coupler 106. The ultrasonic vibration element 110 has the form and shape of an ultrasonic surgical blade, although in other embodiments of the invention, the ultrasonic vibration element 110 may take other forms and shapes. The vibration element 110 is acoustically coupled to the transmission coupler 106, so that the ultrasonic energy is transmitted to, and carried by, the vibration element 110. The vibration element 110 undergoes vibratory motion upon receipt of ultrasonic vibrations from the transducer(s) 104. The vibration element 1 10 thereby delivers ultrasonic energy to the contacting tissue, so that desired surgical effects, such as cutting and/or coagulation, can be achieved.

In the present invention, at least one of the ultrasonic transducer 104, the ultrasonic transmission coupler 106, and the ultrasonic vibration element 110, is disposable. By using inexpensive, disposable component parts, the cost of manufacturing and utilizing the ultrasonic surgical system 100 is significantly lowered, as compared to prior art devices.

In some embodiments of the invention, the ultrasonic transducer sheath, and the tubular sheath enclosing the ultrasonic transmission coupler, are also disposable. In one embodiment, the entire ultrasonic surgical system 100 may be disposable, being composed wholly of disposable parts. In this embodiment, each and every one of the ultrasonic transducer 104, the ultrasonic transmission coupler 106, the ultrasonic vibration element 110, and the ultrasonic transducer sheath, are disposable.

In order to manufacture disposable component parts, the appropriate constituent material must be chosen for each disposable component part. In an embodiment in which the ultrasonic surgical system 100 includes a disposable ultrasonic transducer, the ultrasonic transducer may be made of one of the following materials: piezoelectric materials, piezoceramic materials, and nickel. In an embodiment in which the ultrasonic surgical system 100 includes a disposable ultrasonic vibration element, for example a disposable ultrasonic surgical blade, the materials with which the disposable vibration elements may be formed include the following: plastics, ceramics, polymers, polycarbonates, metals, and plastic-metal alloys.

In the present invention, the disposable component parts are not precision-cut. Rather, the disposable component parts are press-fit, or “snapped on” to each other, so as to form the final surgical assembly. For example, in an embodiment in which the ultrasonic surgical system includes an ultrasonic transducer sheath, and a disposable ultrasonic transducer, the transducer is adapted to be press-fit within the transducer sheath. Similarly, in an embodiment in which the ultrasonic surgical system includes a tubular sheath for enclosing the ultrasonic transmission coupler, the transmission coupler is adapted to be press-fit within the tubular sheath.

Alternatively, the disposable component parts may be threaded, so that each disposable component part can be screwed onto its connecting element. Alternatively, the component parts of the surgical system may be adapted to be connected to each other via a spring mechanism.

Because the component parts are disposable, and not precision-cut, the ultrasonic surgical system of the present invention can accommodate a greater tolerance range, as compared to surgical systems having precision-cut components. Rough, rather than precise, tolerances can be accomodated.

An ultrasonic surgical system, such as the system described above in conjunction with FIG. 1, has a resonant frequency that is determined primarily by the assembled length of its components. Although the ultrasonic surgical system 100, which may be viewed as forming an acoustic assembly, may be vibrated at almost any frequency, efficient and useful vibration occurs only when the acoustic assembly is vibrated at its intended resonant frequency. In this case, maximum vibratory motion occurs at the tip of the vibrating element, with relatively little input power from the ultrasonic generator.

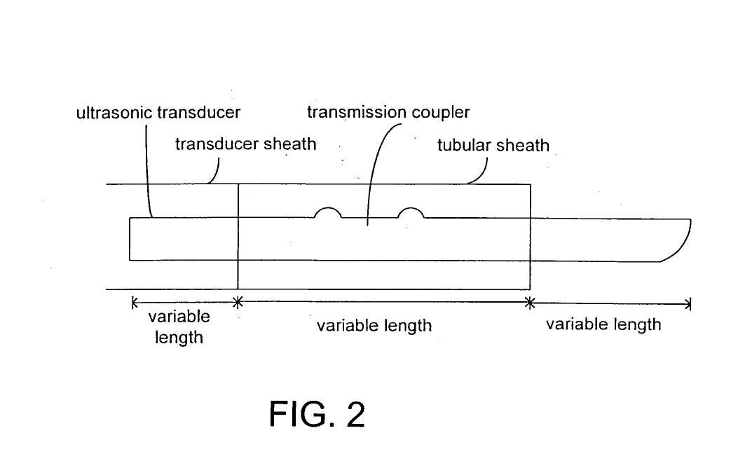

In the present invention, the resonant frequency of the system can be tuned, by varying the lengths of the disposable components until the desired resonant frequency is reached. FIG. 2 provides a schematic illustration of an ultrasonic surgical system whose resonant frequency is tunable by varying the length of one or more of its constituent disposable components.

In order to keep costs down, no specific features (such as specific desired frequencies of vibration) are incorporated by precision-cutting the components. Rather, a constant cross-section material that is suitable for a disposable component part is chosen, then the ultrasonic system is tuned until the desired resonant frequency for the system is reached.

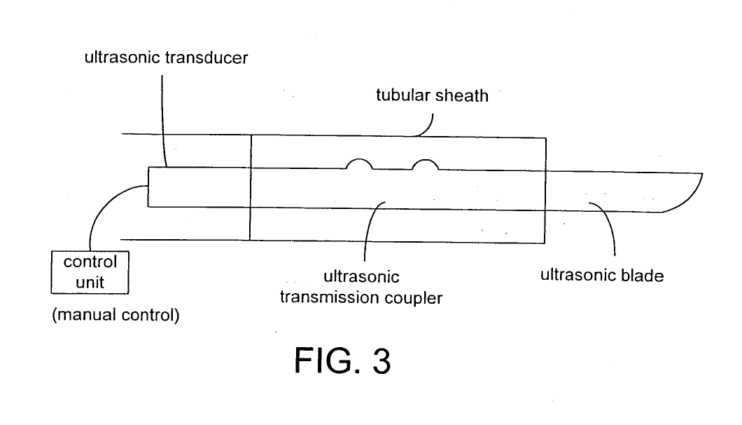

The ultrasonic surgical system of the present invention may include a control unit for controlling the amplitude of the ultrasonic vibrations. Preferably, the control unit is manually controllable, i.e. is a hand-controlled unit. The control unit may also control the frequency and/or duration of the ultrasonic vibrations. FIG. 3 provides a schematic illustration of an ultrasonic surgical system having a control unit for controlling the duration and/or frequency and/or amplitude of the ultrasonic vibrations. As illustrated in FIG. 3, the control unit is connected to the ultrasonic transducer. In one embodiment, the control unit may also be disposable.

In sum, the present invention features an inexpensive ultrasonic surgical system that includes one or more disposable and replaceable component parts that are assembled by press-fitting each component to each other.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. An ultrasonic surgical system, comprising:
a. an ultrasonic transducer for converting electric signals into ultrasonic vibrations;
b. an ultrasonic transmission coupler connected to said transducer so as to receive said ultrasonic vibrations therefrom, said coupler being adapted to transmit said ultrasonic vibrations from a proximal end thereof to a distal end thereof;
c. an ultrasonic vibration element coupled to said distal end of said ultrasonic transmission coupler;
wherein at least one of said ultrasonic transducer, said ultrasonic transmission coupler, and said ultrasonic vibration element is disposable.

2. An ultrasonic surgical system according to claim 1, wherein said ultrasonic transducer is disposable, and wherein said ultrasonic transducer comprises at least one of a piezoelectric material, a piezoceramic material, and nickel.

3. An ultrasonic surgical system according to claim 2, wherein said ultrasonic transducer is adapted to be press-fit onto said ultrasonic transmission coupler.

4. An ultrasonic surgical system according to claim 1, wherein said ultrasonic vibration element comprises a surgical blade.

5. An ultrasonic surgical system according to claim 1, wherein said ultrasonic vibration element is disposable, and wherein said ultrasonic vibration element comprises at least one of a plastic material, a ceramic material, a polymer material, a polycarbonate material, a metal, and a plastic-metal alloy.

6. An ultrasonic surgical system according to claim 5, wherein said ultrasonic surgical system is characterized by a resonant frequency.

7. An ultrasonic surgical system according to claim 1, further comprising an ultrasonic transducer sheath for enclosing said ultrasonic transducer.

8. An ultrasonic surgical system according to claim 7, wherein said ultrasonic transducer sheath is disposable.

9. An ultrasonic surgical system according to claim 8, wherein said ultrasonic transducer is disposable, and wherein said ultrasonic transducer is adapted to be press-fit onto said ultrasonic transducer sheath.

10. An ultrasonic surgical system according to claim 1, further comprising an tubular sheath for enclosing said ultrasonic transmission coupler.

11. An ultrasonic surgical system according to claim 10, wherein said tubular sheath is disposable.

12. An ultrasonic surgical system according to claim 1, further comprising a control unit for controlling at least one of the duration, frequency, and amplitude of said ultrasonic vibrations.

13. An ultrasonic surgical system according to claim 12, wherein said control unit is manually controllable.

14. An ultrasonic surgical system according to claim 13, wherein said control unit is disposable.

15. An ultrasonic surgical system according to claim 1, wherein each of said ultrasonic transducer, said ultrasonic vibration element, and said ultrasonic transmission coupler is disposable.

16. An ultrasonic surgical system according to claim 1, wherein at least one of said ultrasonic transducer, said ultrasonic transmission coupler, and said ultrasonic vibration element is a tunable-length device for which the length is adapted to be varied so as to tune said surgical system to a predetermined resonant frequency.

17. An ultrasonic surgical system according to claim 1, wherein said ultrasonic vibration element is disposable, and is coupled to the ultrasonic transmission coupler via a spring mechanism.

18. An ultrasonic surgical system according to claim 1, wherein at least one of said ultrasonic transducer, said ultrasonic vibration element, and said ultrasonic transmission coupler is fabricated from a constant cross-section material.
